(19) United States
(12) Patent Application Publication
(10) Pub. No.: US 2013/0067987 A1
(43) Pub. Date: Mar. 21, 2013

(54) High sensitivity artificial olfactory system and it's uses

(71) Applicant: Attila Grieb, Calgary (CA)

(72) Inventor: Attila Grieb, Calgary (CA)

(21) Appl. No.: 13/200,076

(22) Filed: Sep. 19, 2011

(51) Int. Cl. G01N 33/497; G01N 37/00

(52) U.S. Cl. 73/23.2

(57) ABSTRACT

High Sensitivity Artificial Olfactory System provide for users an easy way to efficiently detect, identify and track any material(s), plant(s), animal(s), or even person(s), based on the odour properties of these.

This invention is unique by it's special property that's make it able to sensitively and intelligently recognize and make a difference between odour samples, even if their properties are very approaching or mixed with other odour samples.

This invention could be effectively used even where high security is required; to detect, recognize, identify and even track the movement of material(s), animal(s), plant(s) and person(s) Worldwide, in air, water, underground and any other place in Universe, based on the odour/smell properties of these. This invention make easy to keep in track Worldwide the movement of person(s) suspected in crime or terrorist activities, even if the mentioned person travel with false identity documents or the physical appearance has been changed.

### BACKGROUND OF INVENTION

Every year Police, Defence, Border Agencies, Shipping Services, Security and Intelligence Services around the World doing a hard work to identify, detect and to track the movement of persons and materials. The efficiency of today used tools for these purpose is good but not perfect. The High Sensitivity Artificial Olfactory System able to maximize the efficiency of detection, identification and tracking locally and Worldwide of any thing and any being involving internet and satellite communication systems. The High Sensitivity Artificial Olfactory System also can be efficiently used in everyday life uses like search engines, personal security and its applications, research, industry, social media; individually or combined in any sector of use or application based on smell, odour properties of material(s), animal(s), plant(s) person(s), any thing or any being on Earth, underground, water, air and anywhere in Universe.

### SPECIFICATIONS

High Sensitivity Artificial Olfactory System provide for users an easy way to efficiently detect and recognize, and even track the worldwide movement of materials, chemicals or persons based on the odour characteristics and properties of these. High Sensitivity Artificial Olfactory System could be effectively used where high security is required.

The High Sensitivity Artificial Olfactory System could make it easy to keep in track the worldwide movement of persons suspected in crime or terrorist activities, even if the mentioned person travel with false identity documents or the physical appearance(s) has been changed. Also make it possible to keep in track the movement of persons whose protection are a first priority and a critical importance. Odour qualities are individual for everyone, and can be defined and identified only with the right tool.

Also The High Sensitivity Artificial Olfactory System could be used in everyday life uses like search engines, personal security and its applications, social media, industry and research or any combination of these or any sector of use or application based on smell or odour properties of materials, animals or persons on Earth and anywhere in the Universe. Combined with world web or satellite technologies can be used like a powerful and effective tool for High Security and Intelligence Services, Defence, Army, Police, Law Enforcement, Research and Rescue and any other.

The High Sensitivity Artificial Olfactory System can be used individually or combined with any sector of use or any application, where samples data can be collected based on smell or odour characteristics or properties of materials, animals or human; on Earth, air, water, underground and anywhere in Universe where surrounding conditions permit.

Please note that this Utility Patent Application will not contain any drawing or more technical information. The inventor/innovator will keep the right and privacy to develop in his own special way The High Sensitivity Artificial Olfactory System, accordingly do not want disclose further information.

Please note that this Utility Patent Application will not contain any drawing or more technical information. The inventor/innovator will keep the right and privacy to develop in his own special way The High Sensitivity Artificial Olfactory System, accordingly do not want disclose further information.

1. The High Sensitivity Artificial Olfactory System able to gather, process, identify, detect, recognize, track, and transmit information based on odour properties of any material(s), plant(s), animal(s), person(s), any thing or any being. The High Sensitivity Artificial Olfactory System can be used in any sector of use and any location on Earth, in air, water, underground and anywhere in the Universe, where is possible to gather and process information about odour samples and properties of material(s), plant(s), animal(s), person(s), any thing or any being. The High Sensitivity Artificial Olfactory System could be used in everyday life uses like Security, Intelligence Services, Defence, Army, Police, Law Enforcement, Search Engines, Personal Security and its applications, social media, industry, research, Research and Rescue, Border Services and any other use or/and any combination of these or any sector of use or application based on smell or odour properties of material(s), plant(s), animal(s), person(s), any thing and any being on Earth, in air, water, underground and anywhere in the Universe. Combined with world web or satellite technologies can be used like a powerful and effective tool to provide information for users about any thing and any being.
The High Sensitivity Artificial Olfactory System can be used in any sector of use, industry, services, any application, and any action; anywhere where samples data can be collected processed and transmitted, based on smell or odour properties.
